(19) United States
(12) Patent Application Publication
(10) Pub. No.: US 2023/0256068 A1
(43) Pub. Date: Aug. 17, 2023

(54) AMHR2-ED CANCER VACCINE FORMULATIONS

(71) Applicant: The Cleveland Clinic Foundation, Cleveland, OH (US)

(72) Inventors: Vincent K. Tuohy, Broadview Heights, OH (US); Suparna Mazumder, Highland Heights, OH (US); Justin M. Johnson, Cleveland, OH (US)

(21) Appl. No.: 18/169,293

(22) Filed: Feb. 15, 2023

Related U.S. Application Data
(60) Provisional application No. 63/310,731, filed on Feb. 16, 2022.

(51) Int. Cl. A61K 39/00; A61P 37/04; A61K 45/06

(52) U.S. Cl. CPC A61K 39/001102; A61P 37/04; A61K 45/06; A61K 2039/55583; A61K 2039/55566; A61K 2039/55577; A61K 2039/55572

(57) ABSTRACT

Provided herein are compositions, systems, kits, and methods of using a composition comprising at least a portion of an Anti-Mullerian Hormone Receptor Type II extracellular domain (AMHR-ED), and an adjuvant comprising: i) squalene oil, ii) a non-ionic surfactant (e.g., Tween 80), iii) an emulsifier (e.g., sorbitan trioleate), and iv) a buffer (e.g., citrate buffer). In certain embodiments, such compositions are administered to a female subject to treat or prevent ovarian or endometrial cancer (e.g., by inducing expression of anti-AMHR2-ED IgG antibodies by the subject in vivo).

### RELATED APPLICATIONS

The present application claims priority to U.S. Provisional Ser. No. 63/310,731 filed Feb. 16, 2022, which is herein incorporated by reference in its entirety.

### SEQUENCE LISTING

The text of the computer readable sequence listing filed herewith, titled “40282-202_SEQUENCE_LISTING”, created Feb. 15, 2023, having a file size of 10,200 bytes, is hereby incorporated by reference in its entirety.

### FIELD

Provided herein are compositions, systems, kits, and methods of using a composition comprising at least a portion of an Anti-Mullerian Hormone Receptor Type II extracellular domain (AMHR-ED), and an adjuvant comprising: i) squalene oil, ii) a non-ionic surfactant (e.g., Tween 80), iii) an emulsifier (e.g., sorbitan trioleate), and iv) a buffer (e.g., citrate buffer). In certain embodiments, such compositions are administered to a female to treat or prevent ovarian or endometrial cancer (e.g., by inducing expression of anti-AMHR2-ED IgG antibodies by the subject in vivo).

### BACKGROUND

Epithelial ovarian carcinoma (EOC) is the most lethal of all gynecologic malignancies with post-menopausal women, accounting for more than 75% of all cases [1, 2]. An array of non-definitive symptoms associated with EOC onset and a lack of effective biomarkers for early detection often result in late diagnoses at advanced diseased stages with high rates of disease recurrence and poor prognoses following current standard of care [1-7]. Thus, there remains an unmet need for more effective ways to control this disease. The induction of ovarian tumor immunity through vaccination is a promising approach and finds support from the increased overall survival observed in patients whose ovarian tumors are infiltrated by T cells [8].

It has been shown that anti-Müllerian hormone receptor type II (AMHR2) is expressed in ˜92% of primary EOCs, 78% of borderline malignancies, 77-86% of non-EOC ovarian tumors, and 56% of malignant ascites from grades III-IV ovarian cancers [9-12]. AMHR2 is a serine/threonine kinase receptor homologous to type II receptors of the transforming growth factor-beta (TGFβ) superfamily [13]. Anti-Müllerian hormone (AMH) is the cognate ligand of AMHR2, and binding of AMH to the extracellular domain of AMHR2 (AMHR2-ED) signals cell cycle arrest and programmed cell death resulting in regression of the Müllerian ducts during male fetal development, as well as regulation of oocyte development and control of ovarian reserve and fertility in adult females [14, 15]. In adult women, the longest AMHR2 transcript codes for a 573 amino acid protein expressed exclusively in the ovary and consists of a 403 amino acid cytoplasmic domain (AMHR2-CD) that has kinase activity and a 26 amino acid hydrophobic transmembrane domain, both of which show extra-ovarian expression [16]. The long ovarian-specific transcript also codes for a 127 amino acid ligand-binding extracellular domain (AMHR2-ED) expressed exclusively in the human premenopausal ovary [16], and this expression drops to non-immunogenic levels in postmenopausal ovaries [17].

### SUMMARY

Provided herein are compositions, systems, kits, and methods of using a composition comprising at least a portion of an Anti-Mullerian Hormone Receptor Type II extracellular domain (AMHR-ED), and an adjuvant comprising: i) squalene oil, ii) a non-ionic surfactant (e.g., Tween 80), iii) an emulsifier (e.g., sorbitan trioleate), and iv) a buffer (e.g., citrate buffer). In certain embodiments, such compositions are administered to a female to treat or prevent ovarian or endometrial cancer (e.g., by inducing expression of anti-AMHR2-ED IgG antibodies by the subject in vivo).

In some embodiments, provided herein are compositions comprising: a) a polypeptide comprising at least a portion of an Anti-Mullerian Hormone Receptor Type II extracellular domain (AMHR-ED); and b) an adjuvant comprising: i) squalene oil, ii) a non-ionic surfactant, iii) an emulsifier, and iv) a buffer. In some embodiments, the polypeptide is configured to activate CD4+ T cells when administered to a subject (e.g., a human subject, such as a human female subject). In certain embodiments, the polypeptide is configured to induce production of AMHR2-ED specific IgG in vivo when administered to a subject (e.g., a human subject, such as a human female subject).

In other embodiments, provided herein are systems or kits comprising: a) a polypeptide comprising at least a portion of an Anti-Mullerian Hormone Receptor Type II extracellular domain (AMHR-ED) (e.g., from a human); and b) an adjuvant comprising: i) squalene oil, ii) a non-ionic surfactant, iii) an emulsifier, and iv) a buffer. In some embodiments, the polypeptide is configured to activate CD4+ T cells when administered to a subject (e.g., a human subject, such as a human female subject). In certain embodiments, the polypeptide is configured to induce production of AMHR2-ED specific IgG in vivo when administered to a subject (e.g., a human subject, such as a human female subject).

In some embodiments, provided herein are methods of treating an ovarian or endometrial cancer tumor in a female subject, the method comprising: administering to the female subject (e.g., human female) a composition described above and herein. In other embodiments, provided herein are methods of preventing ovarian and/or endometrial cancer in a subject (e.g., a human female subject), the method comprising: administering to the subject a composition described above and herein. In some embodiments, the subject does not have detectable ovarian and/or endometrial cancer.

In certain embodiments, the ovarian cancer tumor is a primary ovarian cancer tumor. In some embodiments, the ovarian cancer tumor is a metastatic tumor. In certain embodiments, the ovarian cancer tumor is an epithelial ovarian cancer (EOC) tumor.

In some embodiments, administration of the composition induces an immune response against the ovarian and/or endometrial cancer tumor in the subject. In some embodiments, administration of the composition induces expression of anti-AMHR2-ED IgG antibodies by the subject.

In some embodiments, the ovarian and/or endometrial cancer tumor expresses AMHR2-ED. In some embodiments, the methods further comprise the step of determining whether the ovarian and/or endometrial cancer tumor expresses AMHR2-ED. In additional embodiments, the methods further comprise the step of determining whether the administration of the composition induces an immune response against the ovarian or endometrial cancer tumor in the subject. In other embodiments, the methods further comprise repeating the administering of the composition to the subject (e.g., once more, twice more, three times more, etc.). In particular embodiments, the adjuvant comprises ADDAVAX™ (InvivoGen) or MF59 (Novartis, Siena), which are commercially available.

In some embodiments, the methods provided herein further comprise the step of administering an additional anti-cancer agent to the subject. In particular embodiments, the additional anti-cancer agent is selected from the group consisting of: paclitaxel, cisplatin, topotecan, gemcitabine, bleomycin, etoposide, carboplatin, docetaxel, doxorubicin, topotecan, cyclophosphamide, trabectedin, olaparib, tamoxifen, letrozole, bevacizumab, an anti-CTLA4 antibody, an anti-PD-1 antibody and an anti-PD-L1 antibody. In some embodiments, the additional agent is an anti-cancer agent. In some embodiments, the anti-cancer agent is paclitaxel, cisplatin, topotecan, gemcitabine, bleomycin, etoposide, carboplatin, docetaxel, doxorubicin, topotecan, cyclophosphamide, trabectedin, olaparib, tamoxifen, letrozole or bevacizumab. In some embodiments, the anti-cancer agent is an immune checkpoint inhibitor. In some embodiments, the immune checkpoint inhibitor is an inhibitor of CTLA4, such as an anti-CTLA4 antibody (e.g., ipilimumab (BMS), tremelimumab (AstraZeneca) and/or KAHR-102 (Kahr Medical)). In some embodiments, the immune checkpoint inhibitor is an inhibitor of PD-1, such as an anti-PD-1 antibody (e.g., nivolumab (BMS), pembrolizumab/lambrolizumab (Merck), pidilizumab (Curetech), AMP-224 (GSK), AMP-514 (AstraZeneca), STI-A1110 (Sorrento) and/or TSR-042 (Tesaro). In some embodiments, the immune checkpoint inhibitor is an inhibitor of PD-L1 and/or PD-L2, such as an anti-PD-L1 and/or an anti-PD-L2 antibody (e.g., RG-7446 (Roche), BMS-936559 (BMS), MEDI-4736 (AstraZeneca), MSB-0020718C (Merck), AUR-012 (Pierre Fabre Med), STI-A1010 (Sorrento)). In some embodiments, the subject has undergone surgery to remove at least part of the ovarian and/or endometrial cancer tumor.

In some embodiments, the emulsifier comprises sorbitan trioleate. In further embodiments, the non-ionic surfactant comprises polysorbate 80, polyglycerol alkyl ethers, glucosyl dialkyl ethers, crownethers, ester-linked surfactants, polyoxyethylene alkyl ethers, Brij, Spans (sorbitan esters) and Tweens (Polysorbates). In additional embodiments, the buffer comprises citrate buffer or phosphate buffered saline. In other embodiments, the pH of the composition is between 5.5 and 6.9.

In certain embodiments, the composition is in the form of an emulsion. In particular embodiments, the emulsion is a nano-emulsification of first and second components, wherein the first component comprises the emulsifier and squalene oil, and the second component comprises the non-ionic surfactant and the buffer. In other embodiments, the emulsifier is present in the first component at about 0.4-0.6 weight/volume (e.g., 0.4%, 0.5%, or 0.6%). In additional embodiments, the squalene oil is present in the first component at about 4-6% weight/volume (e.g., 0.4%, 0.5%, or 0.6%). In some embodiments, the non-ionic surfactant is present in the second component at about 0.4-0.6 weight/volume (e.g., 0.4%, 0.5%, or 0.6%). In additional embodiments, the buffer is present in the second component at about 4-6% weight/volume (e.g., 4%, 5%, or 6%). In particular embodiments, the nano-emulsion is composed of particles with an average size of about 150-170 nm.

In additional embodiments, the adjuvant further comprises at least one of the following: saponins, cholesterol, phospholipids, phosphatidyl choline, and a second buffer. In some embodiments, the second buffer comprises phosphate buffered saline.

In some embodiments, the compositions comprise an oil phase and an aqueous phase. In additional embodiments, the squalene is present at 4.0-5.5% of the oil phase (e.g., 4 . . . 4.3 . . . 4.6 . . . 4.9 . . . 5.2 . . . 5.5%). In other embodiments, the non-ionic surfactant is present about 0.4-0.6% (e.g., 0.4%, 0.5%, or 0.6%) of the aqueous phase. In additional embodiments, the emulsifier is present about 0.4-0.6% of the aqueous phase (e.g., 0.4%, 0.5%, or 0.6%).

In some embodiments, the adjuvant further comprises α-tocopherol. In certain embodiments, the adjuvant further comprises monophosphoryl lipid A. In particular embodiments, the adjuvant further comprises an immunomodulator. In further embodiments, the immunomodulator comprises synthetic trehalose dicorynomycolate.

In certain embodiments, the polypeptide comprises the amino acid sequence in SEQ ID NOS: 7, 8, 9, or 10. In some embodiments, the polypeptide comprises an entire human AMHR-ED or nearly an entire human AMHR2-ED. In some embodiments, the polypeptide comprises at least 20 consecutive amino acids from SEQ ID NO:7. In additional embodiments, the polypeptide comprises at least 40 consecutive amino acids from SEQ ID NO:7. In certain embodiments, the polypeptide comprises at least 60 consecutive amino acids from SEQ ID NO:7. In additional embodiments, the polypeptide comprises at least 100 consecutive amino acids from SEQ ID NO:7. In some embodiments, the polypeptide comprises at least 115 consecutive amino acids from SEQ ID NO:7. In additional embodiments, the polypeptide comprises at least 122 consecutive amino acids from SEQ ID NO:7. In other embodiments, the polypeptide has an amino acid sequence that comprises 100 consecutive amino acids that are at least 80% identical to an amino acid sequence in the extracellular domain of AMHR2.

### BRIEF DESCRIPTION OF THE DRAWINGS

FIG. 1 shows AMHR2 gene expression in mouse EOCs. qRT-PCR analysis of the ID8 and MOVCAR EOC cell lines indicate that both cell lines express AMHR2-ED and AMHR2-CD domains, but the expression of each AMHR2 domain in MOVCAR cells was consistently about twice that of the expression levels measured in ID8 cells. Error bars indicate ±SD.

FIG. 2 shows formulation of the AMHR2-ED vaccine with ADDAVAX™ induces high IgG responses. ELISA analysis shows that the use of ADDAVAX™ as adjuvant when immunizing C57BL/6 female mice resulted in (A) an antigen-specific IgG response against the AMHR2-ED immunogen, and (B) an antigen-specific IgG isotype response similar to that obtained using CFA as adjuvant. Sera were analyzed 8 weeks after immunization using a 1:10,000 dilution. Error bars indicate ±SD.

FIG. 3 shows AMHR2-ED vaccination using ADDAVAX™ as adjuvant is highly effective in inhibiting the growth of mouse EOCs. Prophylactic AMHR2-ED vaccination using ADDAVAX™ as adjuvant resulted in (A) significant inhibition of MOVCAR growth and (B) significant increased overall survival in TgMISIIR-Tag (low) female mice when compared to vaccination with CFA alone or vaccination against AMHR2-ED in CFA. AMHR2-ED vaccination in ADDAVAX™ was also effective in inhibiting the growth of (C) MOVCAR and (D) ID8 tumors when vaccination occurred when tumors became palpable. Error bars=±SD and asterisks indicate significance.

FIG. 4 shows AMHR2-ED vaccination using ADDAVAX™ adjuvant results in T Cell infiltration of murine EOCs. AMHR2-ED vaccination in ADDAVAX™ resulted in infiltrations of CD3+ T cells (arrows, bottom row, left), CD4+ T cells (arrows, bottom row, middle), and CD8+ T cells (arrows, bottom row, right) compared to corresponding EOC tissues from mice vaccinated with ADDAVAX™ alone (arrows, upper row). Tissues are representative of several examined from several different mice. Tissue magnification=20×.

FIG. 5 shows exemplary sequences. Panel A shows an exemplary human AMHR2-ED amino acid sequence (SEQ ID NO:7). Panel B shows an exemplary N terminal truncated human AMHR2-ED amino acid sequence (SEQ ID NO:8). Panel C shows an exemplary C terminal truncated human AMHR2-ED amino acid sequence (SEQ ID NO:9). Panel D shows an exemplary N and C terminal truncated human AMHR2-ED amino acid sequence (SEQ ID NO:10).

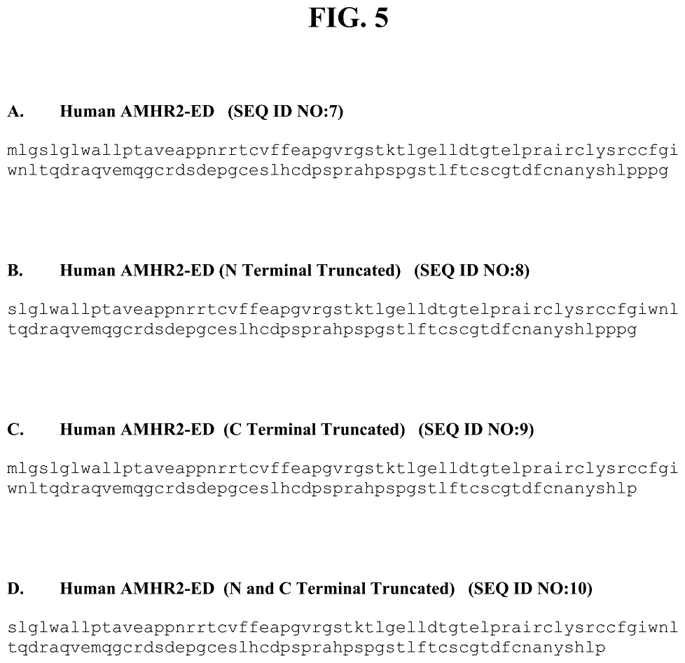

### DEFINITIONS

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent or composition to a subject (e.g., intravenously), and includes, but is not limited to, administering by a medical professional and self-administering.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. In certain embodiments, the compositions herein contain a pharmaceutically-acceptable carrier.

As used herein, the term “subject” means a human or non-human animal selected for treatment or therapy.

The phrases “therapeutically-effective amount” and “effective amount” as used herein means the amount of an agent which is effective for producing the desired therapeutic effect in at least a sub-population of cells in a subject at a reasonable benefit/risk ratio applicable to any medical treatment.

“Treating” a disease in a subject or “treating” a subject having a disease refers to subjecting the subject to a pharmaceutical treatment, e.g., the administration of a vaccine, such that at least one symptom of the disease is decreased or prevented from worsening.

### DETAILED DESCRIPTION

Provided herein are compositions, systems, kits, and methods of using a composition comprising at least a portion of an Anti-Mullerian Hormone Receptor Type II extracellular domain (AMHR-ED), and an adjuvant comprising: i) squalene oil, ii) a non-ionic surfactant (e.g., Tween 80), iii) an emulsifier (e.g., sorbitan trioleate), and iv) a buffer (e.g., citrate buffer). In certain embodiments, such compositions are administered to a female subject (e.g., human) to treat or prevent ovarian or endometrial cancer (e.g., by inducing expression of anti-AMHR2-ED IgG antibodies by the subject in vivo).

In certain embodiments, the adjuvant employed herein is ADDAVAX™ (InvivoGen) and/or an adjuvant chemically identical and/or highly similar to the adjuvant marketed as ADDAVAX™. ADDAVAX™ is a nano-emulsification of 2 components: i) Sorbitan trioleate (0.5% w/v) in squalene oil (5% v/v), and ii) Tween 80 (0.5% w/v) in sodium citrate buffer (10 mM, pH 6.5), where the nano-emulsion is produced using a microfluidizer and filtered through a 0.22-μm filter to remove large droplets and sterilize the final product. The particle size is ˜160 nm.

In certain embodiments, the adjuvant employed herein is MF59 (Novartis, Siena) and/or an adjuvant chemically identical and/or highly similar to the adjuvant marketed as MF59. MF59 is a ˜160 nm particle emulsion comprising: i) Squalene: 9.75 mg; ii) Polysorbate 80: 1.175 mg; iii) Sorbitan trioleate: 1.175 mg; iv) Sodium citrate: 0.66 mg; and v) Citric acid: 0.04 mg.

In certain embodiments, the adjuvant employed herein is Abisco-100 (known as Matrix-M when made to GMP standard), and/or an adjuvant chemically identical and/or highly similar to the adjuvant marketed Abisco-100. Abisco-100 has the following chemical content: purified saponins obtained from a crude extract of the plant Quillaja saponaria Molina; cholesterol from Lanolin and phosphatidyl choline (phospholipid) from fresh egg yolk; in a suspension of nano-sized (40 nm) cage-like particles consisting of the above ingredients, in PBS. Matrix M (or Abisco-100) is composed of a mixture of Matrix A and Matrix C at a ratio of 80:20 to 95:5, preferably 85:15. Matrix A leads to T cell induction and has low toxicity, Matrix C induces antibodies and has some toxicity. Matrix C contains C fraction of QS separation which corresponds to QS21. Fraction A (in Matrix A) corresponds to QS7.

In some embodiments, the adjuvant employed herein is AS03 adjuvant and/or an adjuvant chemically identical and/or highly similar to the adjuvant marketed a AS03 adjuvant. AS03 adjuvant is an emulsion adjuvant composed of an oil phase (10.69 mg squalene, 11.86 mg DL-α-tocopherol) and an aqueous phase (4.86 mg polysorbate 80) each 0.5-mL adult dose. ASO3 has PBS.

In certain embodiments, the adjuvant employed herein is Ribi adjuvant and/or an adjuvant chemically identical and/or highly similar to the adjuvant marketed a Ribi adjuvant. Ribi adjuvant is an oil-in-water emulsion containing 2% squalene-Tween 80-water, 0.5 mg monophosphoryl lipid A (is a low-toxicity derivative of the lipid A region of lipopolysaccharide (LPS)), and 0.5 mg synthetic trehalose dicorynomycolate (an immunomodulator). This adjuvant is in PBS.

In certain embodiments, the compositions provided herein comprise an AMHR2-ED polypeptide and/or an immunologically active fragment of an AMHR2-ED polypeptide. Polypeptides having substantial sequence similarities can cause identical or very similar immune reaction in a host animal. Accordingly, in some embodiments, a derivative, equivalent, variant, fragment, or mutant of AMHR2-ED protein (e.g., as shown in SEQ ID NO:7) or fragment thereof can also be suitable for the methods, compositions and kits provided herein.

In some embodiments, variations or derivatives of the AMHR2-ED polypeptides (e.g., SEQ ID NOs:7-10) are provided herein. The altered polypeptide may have an altered amino acid sequence, for example by conservative substitution, yet still elicits immune responses which react with the unaltered protein antigen, and are considered functional equivalents. As used herein, the term “conservative substitution” denotes the replacement of an amino acid residue by another, biologically similar residue. It is well known in the art that the amino acids within the same conservative group can typically substitute for one another without substantially affecting the function of a protein. According to certain embodiments, the derivative, equivalents, variants, or mutants of the ligand-binding domain of an AMHR2-ED polypeptide are polypeptides that are at least 85% homologous a sequence of the AMHR2-ED protein or fragment thereof. In some embodiments, the homology is at least 90%, at least 95%, or at least 98% (e.g., as compared to SEQ ID NOs:7-10).

In certain aspects, provided herein are pharmaceutical compositions (e.g., a vaccine composition) comprising an AMHR2-ED polypeptide described herein and an adjuvant as described herein. In some embodiments, the composition comprises a pharmaceutically acceptable carrier. In some embodiments, the composition includes a combination of multiple e.g., two or more) AMHR2-ED polypeptides or nucleic acids described herein.

The pharmaceutical compositions disclosed herein may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; or (2) parenteral administration, for example, by subcutaneous, intramuscular, intravenous or epidural injection as, for example, a sterile solution or suspension, or sustained-release formulation.

Methods of preparing these formulations or compositions include the step of bringing into association an AMHR2-ED polypeptide and adjuvants described herein with the carrier and, optionally, one or more accessory ingredients. In general, the formulations are prepared by uniformly and intimately bringing into association an agent described herein with liquid carriers, or finely divided solid carriers, or both, and then, if necessary, shaping the product.

Pharmaceutical compositions suitable for parenteral administration comprise AMHR2-ED polypeptides and adjuvants described herein in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Regardless of the route of administration selected, the agents provided herein, which may be used in a suitable hydrated form, and/or the pharmaceutical compositions disclosed herein, are formulated into pharmaceutically-acceptable dosage forms by conventional methods known to those of skill in the art.

In some embodiments, the pharmaceutical composition described, when administered to a subject, can elicit an immune response against a cell that expresses AMHR2-ED. Such pharmaceutical compositions can be useful as vaccine compositions for prophylactic and/or therapeutic treatment of ovarian or endometrial cancer.

In certain embodiments, compositions provided herein also comprise one or more other agents such as, but not limited to, chemotherapeutic, immunotherapeutic, immunomodulatory and/or anti-angiogenic agents. In some embodiments, the one or more other agents can be a chemotherapeutic agent, naturally occurring or synthetic, for example as described in “Cancer Chemotherapeutic Agents”, American Chemical Society, 1995, W. O. Foye Ed.

In one embodiment, the chemotherapeutic agent is selected from the group consisting of a small molecule receptor antagonists such as vatalanib, SU 11248 or AZD-6474, EGFR or HER2 antagonists such as gefitinib, erlotinib, CI-1033 or Herceptin, antibodies such as bevacizumab, cetuximab, rituximab, DNA alkylating drugs such as cisplatin, oxaliplatin or carboplatin, anthracyclines such as doxorubicin or epirubicin, an antimetabolite such as 5-FU, pemetrexed, gemcitabine or capecitabine, a camptothecin such as irinotecan or topotecan, an anti-cancer drug such as paclitaxel or docetaxel, an epipodophyllotoxin such as etoposide or teniposide, a proteasome inhibitor such as bortezomib or anti-inflammatory drugs such as celecoxib or rofecoxib, optionally in form of the pharmaceutically acceptable salts, in form of the hydrates and/or solvates and optionally in the form of the individual optical isomers, mixtures of the individual enantiomers or racemates thereof.

In another embodiment, the chemotherapeutic agent is selected from the group consisting of a small molecule VEGF receptor antagonist such as vatalanib (PTK-787/ZK222584), SU-5416, SU-6668, SU-11248, SU-14813, AZD-6474, AZD-2171, CP-547632, CEP-7055, AG-013736, IM-842 or GW-786034, a dual EGFR/HER2 antagonist such as gefitinib, erlotinib, CI-1033 or GW-2016, an EGFR antagonist such as iressa (ZD-1839), tarceva (OSI-774), PKI-166, EKB-569, HKI-272 or herceptin, an antagonist of the mitogen-activated protein kinase such as BAY-43-9006 or BAY-57-9006, a quinazoline derivative such as 4-[(3-chloro-4-fluorophenyl)amino]-6-{[4-(N,N-dimethylamino)-1-oxo-2-bute-n-1-yl]amino}-7-((S)-tetrahydrofuran-3-yloxy)quinazoline or 4-[(3-chloro-4-fluoro-phenyl)amino]-6-{[4-(homomorpholin-4-yl)-1-oxo-2-bu-ten-1-yl]amino}-7-[(S)-(tetrahydrofuran-3-yl)oxy]-quinazoline, or a pharmaceutically acceptable salt thereof, a protein kinase receptor antagonist which is not classified under the synthetic small molecules such as atrasentan, rituximab, cetuximab, Avastin™ (bevacizumab), IMC-1C11, erbitux (C-225), DC-101, EMD-72000, vitaxin, imatinib, a protein tyrosine kinase inhibitor which is a fusion protein such as VEGFtrap, an alkylating agent or a platinum compound such as melphalan, cyclophosphamide, an oxazaphosphorine, cisplatin, carboplatin, oxaliplatin, satraplatin, tetraplatin, iproplatin, mitomycin, streptozocin, carmustine (BCNU), lomustine (CCNU), busulfan, ifosfamide, streptozocin, thiotepa, chlorambucil, a nitrogen mustard such as mechlorethamine, an ethyleneimine compound, an alkylsulphonate, daunorubicin, doxorubicin (adriamycin), liposomal doxorubicin (doxil), epirubicin, idarubicin, mitoxantrone, amsacrine, dactinomycin, distamycin or a derivative thereof, netropsin, pibenzimol, mitomycin, CC-1065, a duocarmycin, mithramycin, chromomycin, olivomycin, a phtalanilide such as propamidine or stilbamidine, an anthramycin, an aziridine, a nitrosourea or a derivative thereof, a pyrimidine or purine analogue or antagonist or an inhibitor of the nucleoside diphosphate reductase such as cytarabine, 5-fluorouracile (5-FU), pemetrexed, tegafur/uracil, uracil mustard, fludarabine, gemcitabine, capecitabine, mercaptopurine, cladribine, thioguanine, methotrexate, pentostatin, hydroxyurea, or folic acid, a phleomycin, a bleomycin or a derivative or salt thereof, CHPP, BZPP, MTPP, BAPP, liblomycin, an acridine or a derivative thereof, a rifamycin, an actinomycin, adramycin, a camptothecin such as irinotecan (camptosar) or topotecan, an amsacrine or analogue thereof, a tricyclic carboxamide, an histonedeacetylase inhibitor such as SAHA, MD-275, trichostatin A, CBHA, LAQ824, or valproic acid, an anti-cancer drug from plants such as paclitaxel (taxol), docetaxel or taxotere, a vinca alkaloid such as navelbine, vinblastin, vincristine, vindesine or vinorelbine, a tropolone alkaloid such as colchicine or a derivative thereof, a macrolide such as maytansine, an ansamitocin or rhizoxin, an antimitotic peptide such as phomopsin or dolastatin, an epipodophyllotoxin or a derivative of podophyllotoxin such as etoposide or teniposide, a steganacin, an antimitotic carbamate derivative such as combretastatin or amphetinile, procarbazine, a proteasome inhibitor such as bortezomib, an enzyme such as asparaginase, pegylated asparaginase (pegaspargase) or a thymidine-phosphorylase inhibitor, a gestagen or an estrogen such as estramustine (T-66) or megestrol, an anti-androgen such as flutamide, casodex, anandron or cyproterone acetate, an aromatase inhibitor such as aminoglutethimide, anastrozole, formestane or letrozole, a GNrH analogue such as leuprorelin, buserelin, goserelin or triptorelin, an anti-estrogen such as tamoxifen or its citrate salt, droloxifene, trioxifene, raloxifene or zindoxifene, a derivative of 17.beta.-estradiol such as ICI 164,384 or ICI 182,780, aminoglutethimide, formestane, fadrozole, finasteride, ketoconazole, a LH-RH antagonist such as leuprolide, a steroid such as prednisone, prednisolone, methylprednisolone, dexamethasone, budenoside, fluocortolone or triamcinolone, an interferon such as interferon-beta, an interleukin such as IL-10 or IL-12, an anti-TNFalpha antibody such as etanercept, an immunomodulatory drug such as thalidomide, its R- and S-enantiomers and its derivatives, or revimid (CC-5013), a leukotrien antagonist, mitomycin C, an aziridoquinone such as BMY-42355, AZQ or EO-9, a 2-nitroimidazole such as misonidazole, NLP-1 or NLA-1, a nitroacridine, a nitroquinoline, a nitropyrazoloacridine, a “dual-function” nitro aromatic such as RSU-1069 or RB-6145, CB-1954, a N-oxide of nitrogen mustard such as nitromin, a metal complex of a nitrogen mustard, an anti-CD3 or anti-CD25 antibody, a tolerance induction agent, a biphosphonate or derivative thereof such as minodronic acid or its derivatives (YM-529, Ono-5920, YH-529), zoledronic acid monohydrate, ibandronate sodium hydrate or clodronate disodium, a nitroimidazole such as metronidazole, misonidazole, benzonidazol or nimorazole, a nitroaryl compound such as RSU-1069, a nitroxyl or N-oxide such as SR-4233, an halogenated pyrimidine analogue such as bromodeoxyuridine, iododeoxyuridine, a thiophosphate such as WR-272 1, a photo-chemically activated drug such as porfimer, photofrin, a benzoporphyrin derivative, a pheophorbide derivative, merocyanine 540 (MC-540) or tin etiopurpurin, an ant-template or an anti-sense RNA or DNA such as oblimersen, a non-steroidal inflammatory drug such as acetylsalicyclic acid, mesalazin, ibuprofen, naproxen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluorophen, indomethacin, sulindac, tolmetin, zomepirac, nabumetone, diclofenac, fenclofenac, alclofenac, bromfenac, ibufenac, aceclofenac, acemetacin, fentiazac, clidanac, etodolac, oxpinac, mefenamic acid, meclofenamic acid, flufenamic acid, nifluminic acid, tolfenamic acid, diflunisal, flufenisal, piroxicam, tenoxicam, lomoxicam, nimesulide, meloxicam, celecoxib, rofecoxib, or a pharmaceutically acceptable salt of a non-steroidal inflammatory drug, a cytotoxic antibiotic, an antibody targeting the surface molecules of cancer cells such as apolizumab or 1D09C3, an inhibitor of metalloproteinases such as TIMP-1 or TIMP-2, Zinc, an inhibitor of oncogenes such as P53 and Rb, a complex of rare earth elements such as the heterocyclic complexes of lanthanides, a photo-chemotherapeutic agent such as PUVA, an inhibitor of the transcription factor complex ESX/DRIP130/Sur-2, an inhibitor of HER-2 expression, such as the heat shock protein HSP90 modulator geldanamycin and its derivative 17-allylaminogeldanamycin or 17-AAG, or a therapeutic agent selected from IM-842, tetrathiomolybdate, squalamine, combrestatin A4, TNP-470, marimastat, neovastat, bicalutamide, abarelix, oregovomab, mitumomab, TLK-286, alemtuzumab, ibritumomab, temozolomide, denileukin diftitox, aldesleukin, dacarbazine, floxuridine, plicamycin, mitotane, pipobroman, plicamycin, tamoxifen and testolactone. Preferred compounds include small molecule VEGF receptor antagonist such as vatalanib (PTK-787/ZK222584), SU-5416, SU-6668, SU-11248, SU-14813, AZD-6474, EGFR/HER2 antagonists such as CI-1033 or GW-2016, an EGFR antagonist such as iressa (gefitinib, ZD-1839), tarceva (erlotinib, OSI-774), PKI-166, EKB-569, HKI-272 or herceptin, an antagonist of the mitogen-activated protein kinase such as BAY-43-9006 or BAY-57-9006, atrasentan, rituximab, cetuximab, Avastin™ (bevacizumab), IMC-1C11, erbitux (C-225), DC-101, EMD-72000, vitaxin, imatinib, an alkylating agent or a platinum compound such as melphalan, cyclophosphamide, cisplatin, carboplatin, oxaliplatin, satraplatin, daunorubicin, doxorubicin (adriamycin), liposomal doxorubicin (doxil), epirubicin, idarubicin, a pyrimidine or purine analogue or antagonist or an inhibitor of the nucleoside diphosphate reductase such as cytarabine, 5-fluorouracile (5-FU), pemetrexed, tegafur/uracil, gemcitabine, capecitabine, mercaptopurine, methotrexate, an anti-cancer drug such as paclitaxel (taxol) or docetaxel, a vinca alkaloid such as navelbine, vinblastin, vincristin, vindesine or vinorelbine, an antimitotic peptide such as dolastatin, an epipodophyllotoxin or a derivative of podophyllotoxin such as etoposide or teniposide, a non-steroidal inflammatory drug such as meloxicam, celecoxib, rofecoxib, an antibody targeting the surface molecules of cancer cells such as apolizumab or ID09C3 or the heat shock protein HSP90 modulator geldanamycin and its derivative 17-allylaminogeldanamycin or 17-AAG.

In another embodiment, the chemotherapeutic agent is selected from the group consisting of compounds interacting with or binding tubulin, synthetic small molecule VEGF receptor antagonists, small molecule growth factor receptor antagonists, inhibitors of the EGF receptor and/or VEGF receptor and/or integrin receptors or any other protein tyrosine kinase receptors which are not classified under the synthetic small-molecules, inhibitors directed to EGF receptor and/or VEGF receptor and/or integrin receptors or any other protein tyrosine kinase receptors, which are fusion proteins, compounds which interact with nucleic acids, and which are classified as alkylating agents or platinum compounds, compounds which interact with nucleic acids and which are classified as anthracyclines, as DNA intercalators or as DNA cross-linking agents, including DNA minor-groove binding compounds, anti-metabolites, naturally occurring, semi-synthetic or synthetic bleomycin type antibiotics, inhibitors of DNA transcribing enzymes, and especially the topoisomerase I or topoisomerase II inhibitors, chromatin modifying agents, mitosis inhibitors, anti-mitotic agents, cell-cycle inhibitors, proteasome inhibitors, enzymes, hormones, hormone antagonists, hormone inhibitors, inhibitors of steroid biosynthesis, steroids, cytokines, hypoxia-selective cytotoxins, inhibitors of cytokines, lymphokines, antibodies directed against cytokines, oral and parenteral tolerance induction agents, supportive agents, chemical radiation sensitizers and protectors, photo-chemically activated drugs, synthetic poly- or oligonucleotides, optionally modified or conjugated, non-steroidal anti-inflammatory drugs, cytotoxic antibiotics, antibodies targeting the surface molecules of cancer cells, antibodies targeting growth factors or their receptors, inhibitors of metalloproteinases, metals, inhibitors of oncogenes, inhibitors of gene transcription or of RNA translation or protein expression, complexes of rare earth elements, and photo-chemotherapeutic agents.

In other embodiments, the chemotherapeutic agent is selected from the group consisting of paclitaxel (taxol), docetaxel, a vinca alkaloid such as navelbine, vinblastin, vincristine, vindesine or vinorelbine, an alkylating agent or a platinum compound such as melphalan, cyclophosphamide, an oxazaphosphorine, cisplatin, carboplatin, oxaliplatin, satraplatin, tetraplatin, iproplatin, mitomycin, streptozocin, carmustine (BCNU), lomustine (CCNU), busulfan, ifosfamide, streptozocin, thiotepa, chlorambucil, a nitrogen mustard such as mechlorethamine, an immunomodulatory drug such as thalidomide, its R- and S-enantiomers and its derivatives, or revimid (CC-5013)), an ethyleneimine compound, an alkylsulphonate, daunorubicin, doxorubicin (adriamycin), liposomal doxorubicin (doxil), epirubicin, idarubicin, mitoxantrone, amsacrine, dactinomycin, distamycin or a derivative thereof, netropsin, pibenzimol, mitomycin, CC-1065, a duocarmycin, mithramycin, chromomycin, olivomycin, a phtalanilide such as propamidine or stilbamidine, an anthramycin, an aziridine, a nitrosourea or a derivative thereof, a pyrimidine or purine analogue or antagonist or an inhibitor of the nucleoside diphosphate reductase such as cytarabine, 5-fluorouracile (5-FU), uracil mustard, fludarabine, gemcitabine, capecitabine, mercaptopurine, cladribine, thioguanine, methotrexate, pentostatin, hydroxyurea, or folic acid, an acridine or a derivative thereof, a rifamycin, an actinomycin, adramycin, a camptothecin such as irinotecan (camptosar) or topotecan, an amsacrine or analogue thereof, a tricyclic carboxamide, an histonedeacetylase inhibitor such as SAHA, MD-275, trichostatin A, CBHA, LAQ824, or valproic acid, a proteasome inhibitor such as bortezomib, a small molecule VEGF receptor antagonist such as vatalanib (PTK-787/ZK222584), SU-5416, SU-6668, SU-11248, SU-14813, AZD-6474, AZD-2171, CP-547632, CEP-7055, AG-013736, IM-842 or GW-786034, an antagonist of the mitogen-activated protein kinase such as BAY-43-9006 or BAY-57-9006, a dual EGFR/HER2 antagonist such as gefitinib, erlotinib, CI-1033 or GW-2016, an EGFR antagonist such as iressa (ZD-1839), tarceva (OSI-774), PKI-166, EKB-569, HKI-272 or herceptin, a quinazoline derivative such as 4-[(3-chloro-4-fluorophenyl)amino]-6-{[-4-(N,N-dimethylamino)-1-oxo-2-but-1-en-1-yl]amino}-7-((S)-tetrahydrofuran-3-yloxy)-quinazoline or 4-[(3-chloro-4-fluoro-phenyl)amino]-6-{[4-(homomorpholin-4-yl)-1-oxo-2-bu-ten-1-yl]amino}-7-[(S)-(tetrahydrofuran-3-yl)oxy]-quinazoline, or a pharmaceutically acceptable salt thereof, an inhibitor of the transcription factor complex ESX/DRIP130/Sur-2, an inhibitor of HER-2 expression, such as the heat shock protein HSP90 modulator geldanamycin and its derivative 17-allylaminogeldanamycin or 17-AAG, a protein kinase receptor antagonist which is not classified under the synthetic small molecules such as atrasentan, rituximab, cetuximab, Avastin™ (bevacizumab), IMC-1C11, erbitux (C-225), DC-101, EMD-72000, vitaxin, imatinib, and an antibody targeting the surface molecules of cancer cells such as apolizumab or 1D09C3.

In some embodiments, the anti-cancer agent is an immune checkpoint inhibitor. In some embodiments, the immune checkpoint inhibitor is an inhibitor of CTLA4, such as an anti-CTLA4 antibody (e.g., ipilimumab (BMS), tremelimumab (AstraZeneca) and/or KAHR-102 (Kahr Medical)). In some embodiments, the immune checkpoint inhibitor is an inhibitor of PD-1, such as an anti-PD-1 antibody (e.g., nivolumab (BMS), pembrolizumab/lambrolizumab (Merck), pidilizumab (Curetech), AMP-224 (GSK), AMP-514 (AstraZeneca), STI-A1110 (Sorrento) and/or TSR-042 (Tesaro). In some embodiments, the immune checkpoint inhibitor is an inhibitor of PD-L1 and/or PD-L2, such as an anti-PD-L1 and/or an anti-PD-L2 antibody (e.g., RG-7446 (Roche), BMS-936559 (BMS), MEDI-4736 (AstraZeneca), MSB-0020718C (Merck), AUR-012 (Pierre Fabre Med), STI-A1010 (Sorrento)).

In certain aspects, provided herein are methods for treating or preventing ovarian or endometrial cancer and/or for inducing an immune response against an ovarian or endometrial cancer tumor expressing AMHR2-ED. In certain embodiments, the method comprises administering to a subject a pharmaceutical composition described herein. In some embodiments, the ovarian or endometrial cancer tumor is a primary tumor. In some embodiments, the ovarian or endometrial cancer tumor is a metastatic tumor. In some embodiments, the ovarian cancer tumor is an epithelial ovarian cancer (EOC) tumor. In some embodiments, the ovarian cancer tumor expresses AMHR2-ED.

The methods described herein can be used to treat any subject in need thereof. As used herein, a “subject in need thereof” includes any subject who has ovarian or endometrial cancer, who has had ovarian or endometrial cancer and/or who is predisposed to ovarian or endometrial cancer. For example, in some embodiments, the subject has an ovarian or endometrial cancer tumor (e.g., an ovarian cancer tumor expressing AMHR2-ED). In some embodiments, the subject has undergone surgery to remove at least part of an ovarian or endometrial cancer tumor. In some embodiments, the subject is predisposed to ovarian cancer due to having a BRCA1 or BRCA2 gene mutation in her genome that predisposes the subject to ovarian cancer. In some embodiments, the subject is predisposed to ovarian cancer due to having a TP53 gene mutation, a CHEK2 gene mutation, a RAD51 gene mutation, a BRIP1 gene mutation, and/or a PALB2 gene mutation. In some embodiments, the subject has a family history of ovarian cancer.

The pharmaceutical compositions disclosed herein may be delivered by any suitable route of administration, including orally and parenterally. In certain embodiments the pharmaceutical compositions are delivered generally (e.g., via oral or parenteral administration). In some embodiments, the pharmaceutical compositions are delivered intravenously. In some embodiments, the pharmaceutical composition is delivered intramuscularly. In some embodiments, the pharmaceutical composition is delivered subcutaneously.

The dosage of the subject agent may be determined by reference to the plasma concentrations of the agent. For example, the maximum plasma concentration (Cmax) and the area under the plasma concentration-time curve from time 0 to infinity (AUC (0-4)) may be used. Dosages include those that produce the above values for Cmax and AUC (0-4) and other dosages resulting in larger or smaller values for those parameters.

Actual dosage levels of the active ingredients in the pharmaceutical compositions may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could prescribe and/or administer doses of the agents employed in the pharmaceutical composition at levels lower than that required in order to achieve the desired therapeutic effect and gradually increase the dosage until the desired effect is achieved.

In general, a suitable daily dose of an agent described herein will be that amount of the agent which is the lowest dose effective to produce a therapeutic effect. Such an effective dose will generally depend upon the factors described above.

In one aspect, provided herein is a method of eliciting in a subject an immune response to a cell that expresses AMHR2-ED (e.g., an ovarian cancer tumor cell). The method comprises: administering to the subject a pharmaceutical composition described herein, wherein the pharmaceutically acceptable composition, when administered to the subject, elicits an immune response to the cell that expresses AMHR2-ED. Generally, the immune response can include a humoral immune response, a cell-mediated immune response, or both.

A humoral response can be determined by a standard immunoassay for antibody levels in a serum sample from the subject receiving the pharmaceutical composition. A cellular immune response is a response that involves T cells and can be determined in vitro or in vivo. For example, a general cellular immune response can be determined as the T cell proliferative activity in cells (e.g., peripheral blood leukocytes (PBLs)) sampled from the subject at a suitable time following the administering of a pharmaceutically acceptable composition. Following incubation of e.g., PBMCs with a stimulator for an appropriate period, [³H]thymidine incorporation can be determined. The subset of T cells that is proliferating can be determined using flow cytometry.

In certain aspects, the methods provided herein include administering to both human and non-human mammals. Veterinary applications also are contemplated. In some embodiments, the subject can be any living female organism in which an immune response can be elicited. Examples of subjects include, without limitation, humans, livestock, dogs, cats, mice, rats, and transgenic species thereof.

In certain embodiments, the subject has a history of ovarian or endometrial cancer and has been administered another mode of therapy. The other therapy may have included e.g., surgical resection, radiotherapy, chemotherapy, and other modes of immunotherapy whereby as a result of the other therapy, the subject presents no clinically measurable tumor. However, the subject can be one determined to be at risk for recurrence or progression of the cancer, either near the original tumor site, or by metastases. Such subjects can be further categorized as high-risk and low-risk subjects. The subdivision can be made on the basis of features observed before or after the initial treatment. These features are known in the clinical arts, and are suitably defined for each different cancer. Features typical of high risk subgroups are those in which the tumor has invaded neighboring tissues, or which show involvement of lymph nodes. Thus, for example, a pharmaceutical composition described herein can be administered to the subject to elicit an anti-cancer response primarily as a prophylactic measure against recurrence.

In some embodiments, the pharmaceutical composition can be administered at any time that is appropriate. For example, the administering can be conducted before or during traditional therapy of a subject having an ovarian or endometrial cancer tumor, and continued after the tumor becomes clinically undetectable. The administering also can be continued in a subject showing signs of recurrence.

In some embodiments, the pharmaceutical composition can be administered in a therapeutically or a prophylactically effective amount. Administering the pharmaceutical composition to the subject can be carried out using known procedures, and at dosages and for periods of time sufficient to achieve a desired effect.

In some embodiments, the pharmaceutical composition can be administered to the subject at any suitable site, for example a site that is distal to or proximal to a primary tumor. The route of administering can be parenteral, intramuscular, subcutaneous, intradermal, intraperitoneal, intranasal, intravenous (including via an indwelling catheter), via an afferent lymph vessel, or by any other route suitable in view of the neoplastic disease being treated and the subject's condition. Preferably, the dose will be administered in an amount and for a period of time effective in bringing about a desired response, be it eliciting the immune response or the prophylactic or therapeutic treatment of the neoplastic disease and/or symptoms associated therewith.

The pharmaceutically acceptable composition can be given subsequent to, preceding, or contemporaneously with other therapies including therapies that also elicit an immune response in the subject. For example, the subject may previously or concurrently be treated by chemotherapy, radiation therapy, and other forms of immunotherapy, such other therapies preferably provided in such a way so as not to interfere with the immunogenicity of the compositions described herein.

Administering can be properly timed by the care giver (e.g., physician, veterinarian), and can depend on the clinical condition of the subject, the objectives of administering, and/or other therapies also being contemplated or administered. In some embodiments, an initial dose can be administered, and the subject monitored for an immunological and/or clinical response. Suitable means of immunological monitoring include using patient's peripheral blood lymphocyte (PBL) as responders and neoplastic cells as stimulators. An immunological reaction also can be determined by a delayed inflammatory response at the site of administering. One or more doses subsequent to the initial dose can be given as appropriate, typically on a monthly, semimonthly, or preferably a weekly basis, until the desired effect is achieved. Thereafter, additional booster or maintenance doses can be given as required, particularly when the immunological or clinical benefit appears to subside.

### EXAMPLES

### Example 1

### Ovarian Cancer Vaccine Formulation Using ADDAVAX™ as Adjuvant Induces Significant Inhibition of Murine Epithelial Ovarian Carcinoma Growth

Epithelial ovarian carcinoma (EOC) is the most lethal of all human gynecologic malignancies. It has previously been shown that a single vaccination of female C57BL/6 mice with the extracellular domain of anti-Müllerian hormone receptor II (AMHR2-ED) in an emulsion containing complete Freund's adjuvant (CFA) provides significant prevention and therapy against murine EOCs. It was found that the tumor immunity is mediated by AMHR2-ED-specific IgG that induces EOC cell death prominently through a caspase-3-dependent apoptotic signaling cascade. However, despite the well-established usefulness of CFA as the “gold standard” adjuvant in proof-of-principle animal studies, its use in human clinical vaccination is precluded due to its tendency to induce unresolved granulomas often with painful abscess formation. Since ADDAVAX™ adjuvant has been used safely and successfully to generate high titer antibodies in several human clinical trials, we examined whether vaccination of female C57BL/6 mice with AMHR2-ED in ADDAVAX™ would produce effective tumor immunity. We found that the formulation of AMHR2-ED in ADDAVAX™ induced high serum titers of IgG and a highly significant tumor immunity similar to that obtained using CFA as adjuvant. Our results indicate that vaccination against AMHR2-ED in ADDAVAX™ should provide a novel and effective way to control human EOC.

### 2. Materials and Methods

2.1. Generation of Recombinant Mouse AMHR2-ED. The DNA coding sequence of the entire 125 amino acids of the mature native AMHR2-ED lacking the signal peptide was used to generate the expression construct as previously described [17] (NCBI Reference Sequence: NM 144547.2; Uniprot Q8K592). To optimize protein folding and enhance overall yield, substitutions for native codon sequences were made (Dapcel, Cleveland, Ohio), and the optimized DNA was synthesized de novo to include an N-terminal methionine immediately followed by a FLAG tag, and a C-terminal 6×His tag. The 6×His-tagged AMHR2-ED was purified under denaturing conditions using nickel-nitrilotriacetic acid (Ni-NTA) affinity chromatography (Qiagen, Valencia, Calif.). Prior to use in vitro, the 6×His-tagged AMHR2-ED was further purified by reverse-phase high performance liquid chromatography (HPLC). Levels of endotoxin were determined to be <0.05 endotoxin units (<5 pg) per mg recombinant protein as previously described [17].
2.2. Mice and Murine EOC Cell Lines. TgMISIIR-Tag (DR26) and TgMISIIR-Tag (low) transgenic mice were generously provided by Dr. Denise C. Connolly (Developmental Therapeutics Program, Fox Chase Cancer Center, Philadelphia, Pa.). Female TgMISIIR-Tag (DR26) transgenic mice develop bilateral autochthonous EOCs due to expression of the large T antigen (Tag) of SV40 under control of the AMHR2 promoter [27]. TgMISIIR-Tag (low) transgenic female mice do not develop autochthonous EOCs due to errant transgene insertion, but these mice are immunologically tolerant to SV40-TAg and effectively serve as histocompatible recipients of transplantable mouse ovarian carcinoma (MOVCAR) cells derived from the ascites fluid of TgMISIIR-Tag (DR26) mice [28]. MOVCAR cells were cultured in DMEM (Media Preparation Core, Cleveland Clinic, Cleveland, Ohio) supplemented with 5% fetal bovine serum (HyClone, Logan, Utah), 5% HEPES buffer (Sigma-Aldrich, St. Louis, Mo.), 2 mM L-glutamine (Thermo Fisher Scientific, Waltham, Mass.), and 1% penicillin/streptomycin (Invitrogen, Carlsbad, Calif.). Colonies of transgenic mice were established and maintained by breeding male transgenic mice with wild-type syngeneic C57BL/6 females (Jackson Laboratory, Bar Harbor, Me.). The MOVCAR cells were used to develop EOCs by subcutaneous injection into histocompatible TgMISIIR-Tag (low) transgenic female mice. ID8 mouse ovarian surface epithelial cells (MOSEC) were obtained commercially (Sigma-Aldrich) and cultured in DMEM (Media Preparation Core) containing 4% fetal bovine serum (HyClone), 2 mM L-glutamine (Thermo Fisher Scientific), 1% penicillin/streptomycin (Invitrogen), and insulin-transferrin-sodium selenite media supplement (Sigma-Aldrich). ID8 cells develop EOCs when injected subcutaneously into C57BL/6 female mice [29].
2.3. Transplantable Ovarian Tumors and Treatments. MOVCAR cells (4×10⁶ cells) and ID8 cells (5×10⁶ cells) were suspended in PBS for subcutaneous inoculation in a total volume of 100 μL in the left dorsal flank of female TgMISIIR-Tag (low) and female C57BL/6 mice, respectively. For CFA-based formulation, mice were immunized with a single subcutaneous injection in the right abdominal flank with 200 μL of an emulsion containing 100 μg of recombinant mouse AMHR2-ED in 100 μL of sterile double-distilled deionized water (DDDH₂O) and 100 μL of CFA (Difco, Detroit, Mich.) containing 200 μg of Mycobacterium tuberculosis H37Ra. ADDAVAX™ emulsion was purchased from Invivogen (San Diego, Calif.; catalog #vac-adx-10) and 100 μL was suspended with 100 μL of sterile DDDH₂O containing 100 μg of recombinant mouse AMHR2-ED so that the final emulsion volume of 200 μL was injected into the abdominal flanks of female TgMISIIR-Tag (low) or female C57BL/6 mice. For prophylaxis, mice were vaccinated 15 days prior to tumor inoculation. For therapeutic intervention, mice were vaccinated with AMHR2-ED when tumors became palpable. All experiments started when mice were 6-7 weeks old. Tumor growth was assessed regularly using a Vernier caliper, and the endpoint for all experiments involving transplantable tumors was defined by a tumor measurement of 17 mm in any direction.
2.4. Immunohistochemistry. Briefly, CD3+ T cells were immunostained in 5 μm sections of formalin-fixed paraffin-embedded ID8 tumor tissues using a 1:100 dilution of a primary CD3-specific rabbit monoclonal IgG antibody (Abcam, Cambridge, UK; catalog #ab16669) followed by a horse radish peroxidase (HRP)-conjugated goat anti-rabbit IgG secondary antibody (Abcam; catalog #ab214880). CD4+ and CD8+ T cells were immunostained in 5 μm sections of ovarian tumors by incubation with a 1:1000 dilution of a primary CD4-specific rabbit monoclonal IgG antibody (Abcam; catalog #ab183685) or a 1:500 dilution of a primary CD8-specific rabbit monoclonal IgG antibody (Abcam; catalog #ab217344), followed by HRP-conjugated goat anti-rabbit IgG secondary antibody (Abcam; catalog #ab214880). Visualization was achieved using the DAB substrate kit (BD Pharmingen, San Diego, Calif.; catalog #550880), followed by hematoxylin counterstaining and mounting of sections in mounting medium (Richard-Allan Scientific, Kalamazoo, Mich.; catalog #4112) for examination by light microscopy.
2.5. Real-Time Quantitative RT-PCR (qRT-PCR). Tissues were excised and stored frozen in RNAlater Stabilization Solution (Invitrogen; catalog #AM7021). RNA was extracted from each tissue by homogenization in TRIZOL reagent (Invitrogen: catalog #15596026). qRT-PCR was performed using SYBR Green PCR mix (Applied Biosystems, Carlsbad, Calif.; catalog #4309155) with gene-specific primer pairs (Invitrogen) including mouse AMHR2-CD primers: forward—CTGAGCCGCTGTTCCGATTTGA (SEQ ID NO:1); reverse—ATGTTGGGGCGCTTCCTCTCCT (SEQ ID NO:2); mouse AMHR2-ED primers: forward—GCGGGGAAGCACAAAGACACT (SEQ ID NO:3); reverse—CCGGCCATGGGTAAGATTCC (SEQ ID NO:4); mouse β-actin primers: forward—GGT CAT CAC TAT TGG CAA CG (SEQ ID NO:5); reverse—ACG GAT GTC AAC GTC ACA CT (SEQ ID NO:6). Relative gene expression was determined by normalization of each gene of interest to β-actin expression in each sample.
2.6. Enzyme-Linked Immunosorbent Assays (ELISAs). ELISAs were performed as previously described [29]. Briefly, serum samples were taken from vaccinated mice eight weeks after vaccination, and after diluting each serum sample 1:10,000, were added into triplicate wells onto 96-well plates pre-coated with AMHR2-ED or with the control antigens ovalbumin (Sigma-Aldrich; catalog #A7641-250MG), and recombinant mouse β-casein, a protein generated in E. coli in our laboratory in a manner similar to generation of AMHR2-ED [17]. After completing the incubations at each step, absorbances at 405 nm were determined according to manufacturer's instructions as previously described [17]. Isotype-specific serum antibody responses to AMHR2-ED at 1:10,000 dilutions were determined according to manufacturer's instructions using the Mouse Typer Isotyping Panel (Bio-Rad, Hercules, Calif.; catalog #1722051).
2.7. Biostatistical Analysis. Differences between mRNA expression levels were compared using the Student's t-test and two-way ANOVA. Differences between tumor growth curves were compared using two-way ANOVA. Differences in mouse survival were determined by log-rank and chi-square using correlated samples. All experiments were repeated at least three times independently.

### 3. Results

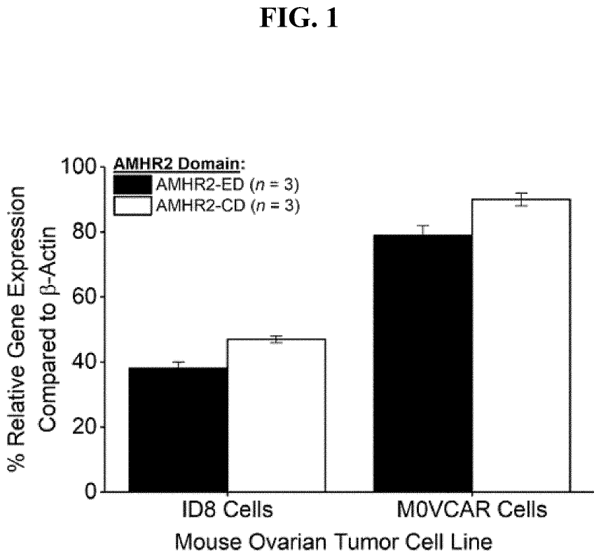

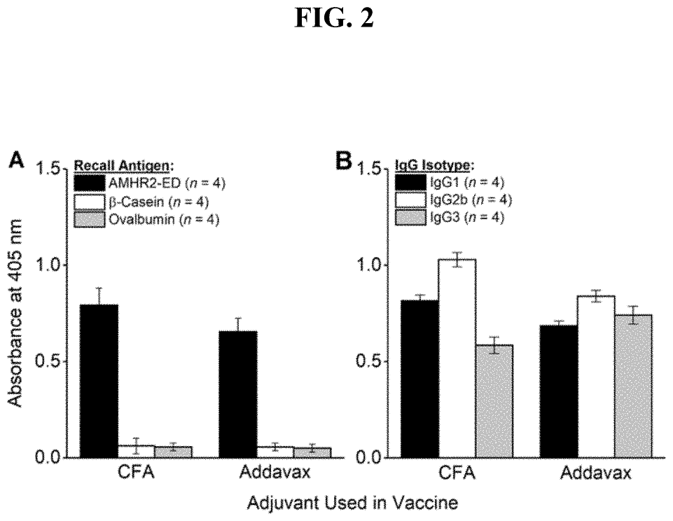

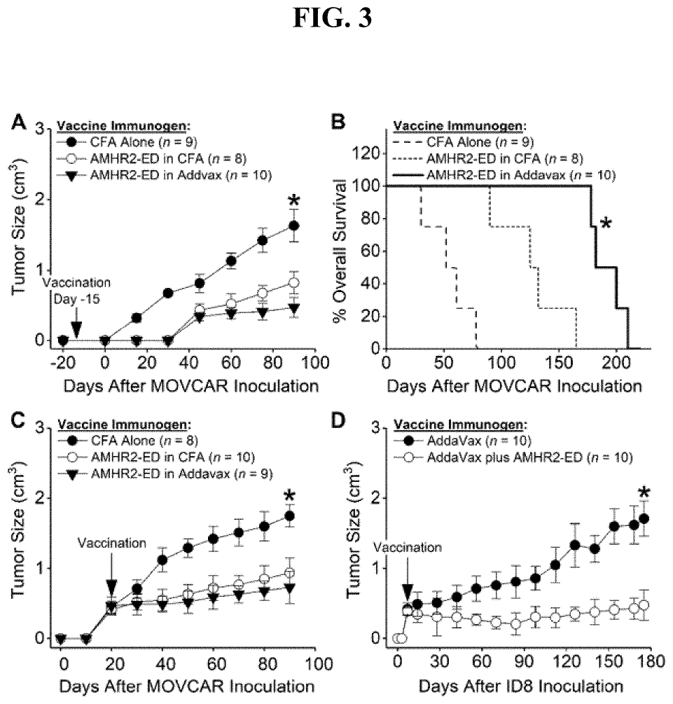

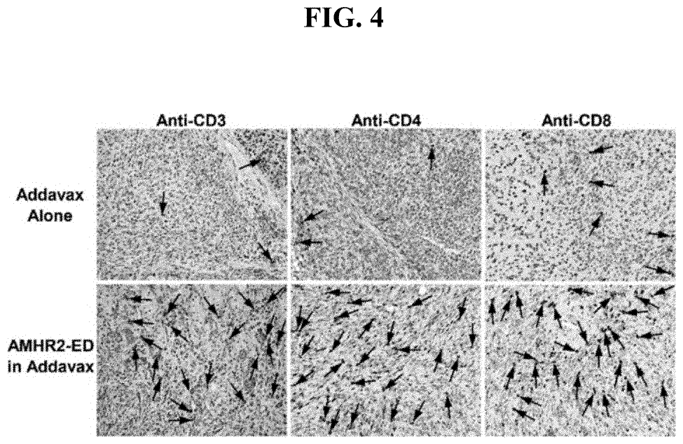

3.1. Elevated AMHR2 Expression in Mouse Ovarian Tumor Cell Lines. We determined the relative gene expression of both AMHR2-ED and AMHR2-CD in ID8 and MOVCAR murine EOC cells. We found that although MOVCAR and ID8 cells express high levels of both AMHR2 domains, the expression levels of each domain was consistently about twice as high in MOVCAR cells compared to ID8 cells (FIG. 1).
3.2. AMHR2-ED Vaccination Formulated Using ADDAVAX™ Adjuvant Induces a Robust IgG Response in Female C57BL/6 Mice. To determine if using ADDAVAX™ as adjuvant would induce a robust IgG response to AMHR2-ED, we immunized 6-7 week-old C57BL/6 female mice with 100 μg of recombinant mouse AMHR2-ED using both CFA and ADDAVAX™ as adjuvants. Eight weeks after immunization the serum IgG response at 1:10,000 dilution was assessed in response to AMHR2-ED and in response to the irrelevant antigens, ovalbumin and recombinant mouse β-casein. We found that the absorbance obtained using ADDAVAX™ as adjuvant was similar to the absorbance obtained using CFA as adjuvant (FIG. 2a). We also found that the IgG isotype profile generated using ADDAVAX™ as adjuvant was also similar to that obtained using CFA as adjuvant (FIG. 2b).
3.3. AMHR2-ED Vaccination Formulated with ADDAVAX™ Effectively Inhibits the Growth of Mouse EOCs. We next examined whether AMHR2-ED vaccination using ADDAVAX™ as adjuvant would compare favorably with the inhibition of murine EOC tumor growth induced when AMHR2-ED vaccination incorporated CFA as adjuvant. We found that prophylactic vaccination against AMHR2-ED using ADDAVAX™ adjuvant 15 days prior to inoculation of MOVCAR cells into TgMISIIR-Tag (low) female mice significantly inhibited the growth of the MOVCAR-derived tumors when compared to vaccination with CFA alone or when compared to vaccination with AMHR2-ED using CFA as adjuvant (FIG. 3a; P<0.0001 for each). Moreover, prophylactic vaccination against AMHR2-ED using ADDAVAX™ as adjuvant provided a statistically significant increase in overall survival when compared to vaccination with CFA alone (FIG. 3b; P<0.009) or when compared to vaccination against AMHR2-ED using CFA as adjuvant (FIG. 3b; P<0.006). We also found that therapeutic vaccination against AMHR2-ED when MOVCAR tumors became palpable was similarly effective whether using CFA or ADDAVAX™ as adjuvant compared to vaccination with CFA alone (FIG. 3c; P<0.0001 for each). However, perhaps the best way to appreciate the efficacy of ADDAVAX™ is most clearly evident when female C57BL/6 mice were vaccinated with either ADDAVAX™ alone or with AMHR2-ED using ADDAVAX™ as adjuvant after the inoculated ID8 EOC cells became a palpable tumors (FIG. 3d; P<0.0001).
3.4. AMHR2-ED Vaccination Using ADDAVAX™ as Adjuvant Results in T Cell Infiltration of Murine EOCs. We next examined the immune cell infiltrates of ID8 tumors 175 days after tumor inoculation and 168 days after AMHR2-ED vaccination using ADDAVAX™ as adjuvant. We observed substantial infiltration of CD3+, CD4+, and CD8+ T cells (FIG. 4). The observed infiltration of CD8+ T cells into the ID8 tumors was rather surprising because we had not previously observed CD8+ T cell infiltration in C57BL/6 female mice vaccinated against AMHR2-ED when CFA was used as adjuvant [17].

The current Example shows that formulation of our AMHR2-ED vaccine with ADDAVAX™ as adjuvant induces a robust IgG response and inhibits the growth of mouse EOCs in a manner similar to, and generally superior to, using CFA as adjuvant. In fact, with some comparisons, ADDAVAX™ seemed to be surprisingly superior to CFA as adjuvant, most notably in inducing a much more enhanced overall survival in the transplantable MOVCAR EOC mouse model and in T cell infiltration of CD8+ T cells in the ID8-derived EOC tumor beds that did not occur in prior studies using CFA as adjuvant [17]. Thus, our data support the substitution of ADDAVAX™ for CFA as an adjuvant in prevention of EOC in women at high genetic risk for developing EOC. Studies have shown that 39%-44% of women with a harmful BRCA1 variant and 11%-17% of women with a harmful BRCA2 variant develop ovarian cancer by 70-80 years of age [30-32]. The availability of such high-risk populations of women with a high incidence of EOC and with the greatest need for an EOC preventive vaccine makes it important to treat women with the adjuvant and AMHR2-ED peptides herein.

### REFERENCES

1. Siegel, et al., Jemal, A. Cancer Statistics, 2017. CA. Cancer J. Clin. 2017, 67, 7-30.
2. Howlader, N.; Noone, A. M.; Krapcho, M.; Miller, D.; Brest, A.; Yu, M.; Ruhl, J.; Tatalovich, Z.; Mariotto, A.; Lewis, D. R.; et al. (eds). SEER Cancer Statistics Review, 1975-2018, National Cancer Institute. Bethesda, Md., https://seer.cancer.gov/csr/1975_2018/, based on November 2020 SEER data submission, posted to the SEER web site, April 2021.
3. Pignata, et al., Treatment of recurrent epithelial ovarian cancer. Cancer 2019; 125, Suppl 24, 4609-4615.
4. Pfisterer, J.; Ledermann, J. A. Management of platinum-sensitive recurrent ovarian cancer. Semin. Oncol. 2006, 33, (2 Suppl 6), S12-16.
5. Fung-Kee-Fung, et al., Optimal chemotherapy treatment for women with recurrent ovarian cancer. Curr. Oncol. 2007, 14, 195-208.
6. Montemorano, L.; Lightfoot, M. D.; Bixel, K. Role of Olaparib as maintenance treatment for ovarian cancer: The evidence to date. Onco. Targets Ther. 2019, 12, 11497-11506.
7. Kubalanza, K.; Konecny, G. E. Mechanisms of PARP inhibitor resistance in ovarian cancer. Curr. Opin. Obstet. Gynecol. 2020, 32, 36-41.
8. Zhang, et al. Intratumoral T cells, recurrence, and survival in epithelial ovarian cancer. N. Engl. J. Med. 2003, 348, 203-213.
9. Masiakos, et al. Human ovarian cancer, cell lines, and primary as-cites cells express the human Mullerian inhibiting substance (MIS) type II receptor, bind, and are re-sponsive to MIS. Clin. Cancer. Res. 1999, 5, 3488-3499.
10. Bakkum-Gamez, et al., Müllerian inhibiting substance type II receptor (MISIIR): a novel, tissue-specific target expressed by gynecologic cancers. Gynecol. Oncol. 2008, 108, 141-148.
11. Song, et al., The expression of Müllerian inhibiting substance/anti-Müllerian hormone type II receptor protein and mRNA in benign, borderline and malignant ovarian neoplasia. Int. J. Oncol. 2009, 34, 1583-1591.
12. Mazumder, et al., Immunotherapy of ovarian cancer with a monoclonal antibody specific for the extracellular domain of anti-Müllerian hormone receptor II. Oncotarget 2020, 11, 1894-1910.
13. Kristensen, et al., Expression of TGF-beta superfamily growth factors, their receptors, the associated SMADs and antagonists in five isolated size-matched populations of pre-antral follicles from normal human ovaries. Mol. Hum. Reprod. 2014, 20, 293-308.
14. Josso, et al., Anti-Müllerian hormone and its receptors. Mol. Cell. Endocrinol. 2001, 179, 25-32.
15. Gruijters, et al., Anti-Müllerian hormone and its role in ovarian function. Mol. Cell. Endocrinol. 2003, 211, 85-90.
16. Uhlen, et al. Towards a knowledge-based Human Protein Atlas. Nat. Biotechnol. 2010, 28, 1248-1250.
17. Mazumder, et al., Primary immunoprevention of epithelial ovarian carcinoma by vaccination against the extracellular domain of anti-Müllerian hormone receptor II. Cancer Prev. Res. (Phila). 2017, 10, 612-624.
18. Tuohy, V. K.; Johnson, J. M.; Mazumder, S. Primary immunoprevention of adult onset cancers by vaccinating against retired tissue-specific self proteins. Semin. Immunol. 2020, 47, 1-7.
19. Chapel, et al., Report of nine cases of accidental injury to man with Freund's complete adjuvant. Clin. Exp. Immunol. 1976, 24, 538-541.
20. Stills, H. F., Jr. Adjuvants and antibody production: dispelling the myths associated with Freund's complete and other adjuvants. ILAR J. 2005, 46, 280-293.
21. Apostólico, et al., Adjuvants: Classification, Modus Operandi, and Licensing. J. Immunol. Res. 2016, 2016, 1459394.
22. Ott, et al., MF59. De-sign and evaluation of a safe and potent adjuvant for human vaccines. Pharm. Biotechnol. 1995, 6, 277-296.
23. Black, S. Safety and effectiveness of MF-59 adjuvanted influenza vaccines in children and adults. Vac-cine 2015, 33, Suppl 2:B3-5.
24. Bernstein, et al. Safety and efficacy of a cytomegalovirus glycoprotein B (gB) vaccine in adolescent girls: A randomized clinical trial. Vaccine 2016, 34, 313-319.
25. Essink, et al., Immunogenicity and safety of MF59-adjuvanted quadrivalent influenza vaccine versus standard and al-ternate B strain MF59-adjuvanted trivalent influenza vaccines in older adults. Vaccine 2020, 38, 242-250.
26. Gray, et al. Vaccine efficacy of ALVAC-HIV and bivalent subtype C gp120-MF59 in adults. N. Engl. J. Med. 2021, 384, 1089-1100.
27. Connolly, et al., Female mice chimeric for expression of the simian virus 40 TAg under control of the MISIIR promoter develop epithelial ovarian cancer. Cancer Res. 2003, 63, 1389-1397.
28. Quinn, et al., Development of a syngeneic mouse model of epithelial ovarian cancer. J. Ovarian Res. 2010, 3, 24.
29. Sakalar, et al., Regulation of murine ovarian epithelial carcinoma by vaccination against the cytoplasmic domain of anti-Müllerian hormone receptor II. J. Immunol. Res. 2015, 2015, 630287.
30. Antoniou, et al. Average risks of breast and ovarian cancer associated with BRCA1 or BRCA2 mutations detected in case series unselected for family history: a combined analysis of 22 studies. Am. J. Hum. Genet. 2003, 72, 1117-1130.
31. Chen, S.; Parmigiani, G. Meta-analysis of BRCA1 and BRCA2 penetrance. J. Clin. Oncol. 2007, 25, 1329-1333.
32. Kuchenbaecker, et al. Risks of breast, ovarian, and contralateral breast cancer for BRCA1 and BRCA2 mutation carriers. JAMA. 2017, 317, 2402-2416.
33. Strasser-Weippl, et al., Suitable trial designs and cohorts for preventive breast cancer agents. Nat. Rev. Clin. Oncol. 2013, 10, 677-687. doi: 10.1038/nrclinonc.2013.174
34. Temkin, et al., Ovarian cancer prevention in high-risk women. Clin. Obstet. Gynecol. 2017, 60, 738-757.
35. National Comprehensive Cancer Network, NCCN clinical practice guidelines in oncology. Ovarian Cancer Including Fallopian Tube Cancer and Primary Peritoneal Cancer, Version 1.2020—Mar. 11, 2020; https://www.nccn.org/professionals/physician_gls/pdf/ovarian.pdf
36. Manoukian, et al., Risk-reducing surgery in BRCA1/BRCA2 mutation carriers: Are there factors associated with the choice? Psychooncology 2019, 28, 1871-1878.

### INCORPORATION BY REFERENCE

All publications, patents, and patent applications mentioned herein are hereby incorporated by reference in their entirety as if each individual publication, patent or patent application was specifically and individually indicated to be incorporated by reference.

We claim:

1. A composition comprising:
a) a polypeptide comprising at least a portion of an Anti-Mullerian Hormone Receptor Type II extracellular domain (AMHR-ED); and
b) an adjuvant comprising:
i) squalene oil,
ii) a non-ionic surfactant,
iii) an emulsifier, and
iv) a buffer.

2. The composition of claim 1, wherein said emulsifier comprises sorbitan trioleate, and/said non-ionic surfactant comprises polysorbate 80.

3. The composition of claim 1 or claim 2, wherein said polypeptide is from a human AMHR-ED.

4. The composition of any one of claims 1-3, wherein said buffer comprises citrate buffer or phosphate buffered saline.

5. The composition of any one of claims 1-4, wherein the pH of said composition is between 5.5 and 6.9.

6. The composition of any one of claims 1-5, wherein said composition is in the form of an emulsion.

7. The composition of claim 6, wherein said emulsion is a nano-emulsification of first and second components, wherein said first component comprises said emulsifier and squalene oil, and said second component comprises said non-ionic surfactant and said buffer.

8. The composition of claim 7, wherein said emulsifier is present in said first component at about 0.4-0.6 weight/volume.

9. The composition of claim 7 or claim 8, wherein said squalene oil is present in said first component at about 4-6% weight/volume.

10. The composition of any one of claims 7-9, wherein said non-ionic surfactant is present in said second component at about 0.4-0.6 weight/volume.

11. The composition of any one of claims 7-10, wherein said buffer is present in said second component at about 4-6% weight/volume.

12. The composition of any one of claims 7-11, wherein said nano-emulsion is composed of particles with an average size of about 150-170 nm.

13. The composition of any one of claims 1-12, wherein said adjuvant further comprises at least one of the following: saponins, cholesterol, phospholipids, phosphatidyl choline, and a second buffer.

14. The composition of any one of claims 1-13, wherein said adjuvant further comprises α-tocopherol.

15. The composition of any one of claims 1-14, wherein said adjuvant further comprises monophosphoryl lipid A.

16. The composition of any one of claims 1-15, wherein said adjuvant further comprises an immunomodulator.

17. The composition of claim 16, wherein said immunomodulator comprises synthetic trehalose dicorynomycolate.

18. The composition of any one of claims 1-17, wherein said polypeptide comprises the amino acid sequence of SEQ ID NOS: 7, 8, 9, or 10.

19. The composition of any one of claims 1-17, wherein said polypeptide comprises an entire human AMHR-ED or nearly an entire human AMHR2-ED.

20. The composition of any one of claims 1-17, wherein said polypeptide comprises at least consecutive amino acids from SEQ ID NO:7.

21. The composition of any one of claims 1-17, wherein said polypeptide comprises at least consecutive amino acids from SEQ ID NO:7.

22. The composition of any one of claims 1-17, wherein said polypeptide comprises at least 60 consecutive amino acids from SEQ ID NO:7.

23. The composition of any one of claims 1-17, wherein said polypeptide comprises at least 100 consecutive amino acids from SEQ ID NO:7.

24. The composition of any one of claims 1-17, wherein said polypeptide comprises at least 115 consecutive amino acids from SEQ ID NO:7.

25. The composition of any one of claims 1-17, wherein said polypeptide comprises at least 122 consecutive amino acids from SEQ ID NO:7.

26. The composition of any one of claims 1-17, wherein the polypeptide has an amino acid sequence that comprises 100 consecutive amino acids that are at least 80% identical to an amino acid sequence in the extracellular domain of AMHR2.

27. The composition of any one of claims 1-26, wherein administration of the composition to a subject activates CD4+ T cells in the subject.

28. The composition of any one of claims 1-27, wherein administration of the composition to a subject induces production of AMHR2 ED-specific IgG in the subject.

29. The composition of claim 27 or 28, wherein the subject is a human female.

30. A method of treating an ovarian or endometrial cancer tumor in a female subject, the method comprising: administering to the female subject a composition of any of claims 1-29.

31. The method of claim 30, wherein the subject has an ovarian cancer tumor and said ovarian cancer tumor is a primary ovarian cancer tumor, or a metastatic tumor.

32. The method of claim 30 or 31, wherein said female subject is a human.

33. The method of any one of claims 30-32, wherein the subject has an ovarian cancer tumor and said ovarian cancer tumor is an epithelial ovarian cancer (EOC) tumor.

34. The method of any one of claims 30-33, wherein administration of said composition induces an immune response against said ovarian and/or endometrial cancer tumor in said subject.

35. The method of any one of claims 30-34, wherein said ovarian and/or endometrial cancer tumor expresses AMHR2-ED.

36. The method of any one of claims 30-35, wherein the method further comprises the step of determining whether said ovarian and/or endometrial cancer tumor expresses AMHR2-ED.

37. The method of any one of claims 30-36, further comprising the step of determining whether the administration of said composition induces an immune response against said ovarian or endometrial cancer tumor in said subject.

38. The method of any one of claims 30-37, further comprising repeating said administering of said composition to said subject.

39. The method of any one of claims 30-38, wherein administration of said composition induces expression of anti-AMHR2-ED IgG antibodies by said subject.

40. The method of any one of claims 30-39, further comprising the step of administering an additional anti-cancer agent to the subject.

41. The method of claim 40, wherein the additional anti-cancer agent is paclitaxel, cisplatin, topotecan, gemcitabine, bleomycin, etoposide, carboplatin, docetaxel, doxorubicin, topotecan, cyclophosphamide, trabectedin, olaparib, tamoxifen, letrozole, bevacizumab, an anti-CTLA4 antibody, an anti-PD-1 antibody or an anti-PD-L1 antibody.

42. The method of claim any one of claims 30-41, wherein the subject has undergone surgery to remove at least part of the ovarian and/or endometrial cancer tumor.

43. A method of preventing ovarian and/or endometrial cancer comprising: administering to a female subject a composition of any of claims 1-29.

44. The method of claim 43, wherein the subject does not have detectable ovarian and/or endometrial cancer.

45. The method of claim 43 or 44, wherein the subject is at elevated risk for developing ovarian and/or endometrial cancer.

46. A system or kit comprising:
a) a polypeptide comprising at least a portion of an Anti-Mullerian Hormone Receptor Type II extracellular domain (AMHR-ED); and
b) an adjuvant comprising:
i) squalene oil,
ii) a non-ionic surfactant,
iii) an emulsifier, and
iv) a buffer.

47. The system or kit of claim 46, wherein said polypeptide is from a human AMHR-ED.


SEQUENCE LISTING (10 sequences)

SEQ ID NO: 1 - DNA, 22 bp, synthetic construct
  source 1..22: mol_type = other DNA
ctgagccgctgttccgatttga

SEQ ID NO: 2 - DNA, 22 bp, synthetic construct
  source 1..22: mol_type = other DNA
atgttggggcgcttcctctcct

SEQ ID NO: 3 - DNA, 21 bp, synthetic construct
  source 1..21: mol_type = other DNA
gcggggaagcacaaagacact

SEQ ID NO: 4 - DNA, 20 bp, synthetic construct
  source 1..20: mol_type = other DNA
ccggccatgggtaagattcc

SEQ ID NO: 5 - DNA, 20 bp, synthetic construct
  source 1..20: mol_type = other DNA
ggtcatcactattggcaacg

SEQ ID NO: 6 - DNA, 20 bp, synthetic construct
  source 1..20: mol_type = other DNA
acggatgtcaacgtcacact

SEQ ID NO: 7 - protein, 127 aa, Homo sapiens
MLGSLGLWALLPTAVEAPPNRRTCVFFEAPGVRGSTKTLGELLDTGTELPRAIRCLYSRCCFGIWNLTQDRAQVEMQGCRDSDEPGCESLHCDPSPRAHPSPGSTLFTCSCGTDFCNANYSHLPPPG

SEQ ID NO: 8 - protein, 124 aa, Homo sapiens
SLGLWALLPTAVEAPPNRRTCVFFEAPGVRGSTKTLGELLDTGTELPRAIRCLYSRCCFGIWNLTQDRAQVEMQGCRDSDEPGCESLHCDPSPRAHPSPGSTLFTCSCGTDFCNANYSHLPPPG

SEQ ID NO: 9 - protein, 124 aa, Homo sapiens
MLGSLGLWALLPTAVEAPPNRRTCVFFEAPGVRGSTKTLGELLDTGTELPRAIRCLYSRCCFGIWNLTQDRAQVEMQGCRDSDEPGCESLHCDPSPRAHPSPGSTLFTCSCGTDFCNANYSHLP

SEQ ID NO: 10 - protein, 121 aa, Homo sapiens
SLGLWALLPTAVEAPPNRRTCVFFEAPGVRGSTKTLGELLDTGTELPRAIRCLYSRCCFGIWNLTQDRAQVEMQGCRDSDEPGCESLHCDPSPRAHPSPGSTLFTCSCGTDFCNANYSHLP